(19) United States
(12) Patent Application Publication
(10) Pub. No.: US 2009/0318795 A1
(43) Pub. Date: Dec. 24, 2009

(54) IMAGING TABLE WITH REPLACEABLE SUPPORT PARTS

(76) Inventor: Remo J. Rossi, Sterling, MA (US)

Correspondence Address: LAW OFFICE OF BRETT N. DORNY, 386 WEST MAIN STREET, SUITE 12A, NORTHBOROUGH, MA, 01532, US

(21) Appl. No.: 12/142,584

(22) Filed: Jun. 19, 2008

(51) Int. Cl. A61B 5/05

(52) U.S. Cl. 600/415

(57) ABSTRACT

An imaging table is disclosed which allows for proper positioning of patients in a face down prone position. The table top includes an access hole towards the head of the table. Various modular components may be positioned within the access hole. The modular components are designed to fit with in the access hole so that a support surface may be above or below the top of the table. A modular component may be reversible, providing a support surface above the table in one position and below the table in another position. A hole may be formed in a support surface to fit the patient's face to ease breathing. A modular component is designed to be inserted into the table top access hole in order to use the table with a continuous, flat surface for treatment and/or imaging procedures in which a face down, prone position is not required.

### BACKGROUND OF THE INVENTION

1. Field of the Invention

The present invention relates to medical imaging tables. More particularly, it relates to a medical imaging table having replaceable parts to allow comfortable patient positioning and proper flexion of the neck and spine for enhanced physician access and improved imaging definition and quality when a patient is in the prone, face down position during procedures.

2. Discussion of Related Art

Currently carbon fiber imaging tables and table tops are manufactured in a variety of configurations based on the clinical requirements of each imaging procedure, as well as doctor preferences and patient comfort. Since different imaging procedures require different patient and/or doctor positions for optimal access to the subject anatomy, the table top size and shape on a specific table model may vary depending upon the type of imaging procedure or procedures that the particular imaging table is designed to facilitate. Additionally, a table top pad or mattress of uniform thickness, and with outer dimensions shaped to coincide with the shape of the table top, is used to provide a cushioned surface for the patient to lie on.

Additionally, positioning a patient in a face down prone position can be problematic. For example, in the field of Pain Management/Spinal Imaging the patient is placed in the prone or face down position so that the doctor can access the spine while imaging. In order to ease the patient's breathing, a pillow or pad may be placed under the patient's forehead, or a c-shaped face cushion similar to those used on a massage table is used to support the patient's head at the perimeter of the face. Some table manufacturers cut holes into the table top to provide facial clearance and breathing access. In this way, the patient's eyes, nose and mouth are positioned over the hole in the table top while the outer perimeter of the face is supported by the table top pad. An additional pad or c-shaped face cushion may be placed on the table top pad to permit elevating the face where necessary.

The other aspect of patient positioning relates to the orientation of the spine which is important to facilitate both access for treatment and optimal imaging views of the subject spinal anatomy. Positioning the patient can be somewhat challenging based on the differences in the “geometry” of people, thin vs. large. Obviously when placed on a flat and stiff table top the location of a persons face relative to their chest and lower abdominal region will vary based on their relative size and girth.

The question then becomes how to both position a patient in such a way on a flat surface so that free breathing may be possible while not compromising the ideal geometry or positioning aspects of the procedure. Some table manufacturers cut holes in the face section of their tops, but as this cut out is co-planar with the top surface, the location of the hole is not necessarily coincident with the location that is optimal for positioning. With a plane surface to support them the position of a patients face will vary in distance to the plane of the top necessitating additional pillows or foam blocks to provide support

Other manufacturers sell cushioned add-on devices that can be placed on the table top and under the patient to help maintain ideal spinal geometry; some of these devices even incorporate a face support cushion. As these devices are placed directly under the spine they are by default directly in the imaging field of view. While they may be made out of component that are radio-translucent, they will still create artifacts and further increase the overall x-ray absorption of the table top.

### SUMMARY OF THE INVENTION

The present invention is a table which is able to properly position patients in a face down prone position. Additionally, it can be used as a standard flat table for patients in other positions. According to an aspect of the invention, a carbon fiber imaging table includes a recess positioned towards one end. According to another aspect of the invention, the imaging table includes a plurality of inserts which fit into the recess. The inserts have various dimensions to allow variable positioning of the patient's head in both planar and angular positions.

According to another aspect of the invention, an insert includes an oval opening to accommodate a patient's face. According to another aspect of the invention, an insert has two sides and is reversible. One side provides a recessed cavity; when reversed, the cavity forms an extension above the other side extends above the surface of the table to support the patient's face. When placed within the recess of the table in one direction, the insert extends above the surface of the table top. When placed in the recess of the table in the opposite direction, the insert provides a recess for the patient's head. According to another aspect of the invention, an insert provides a level surface with the top of the table. According to another aspect of the invention, an insert includes an upper surface which extends above the table top at an angle relative to the top of the table.

### BRIEF DESCRIPTION OF THE DRAWINGS

FIG. 1 is a top view of an imaging table according to an embodiment of the present invention.

FIG. 2 is a top view of an imaging table with an insert according to an embodiment of the present invention.

FIG. 3 is a top view of an imaging table with an insert according to another embodiment of the present invention.

FIG. 4 is a side view of an imaging table with an insert according to another embodiment of the present invention.

FIG. 5 is a side view of an imaging table with an insert according to another embodiment of the present invention.

FIG. 6 is a side view of an imaging table with an insert according to another embodiment of the present invention.

FIG. 7 is a side view of an imaging table with an insert according to another embodiment of the present invention.

### DETAILED DESCRIPTION

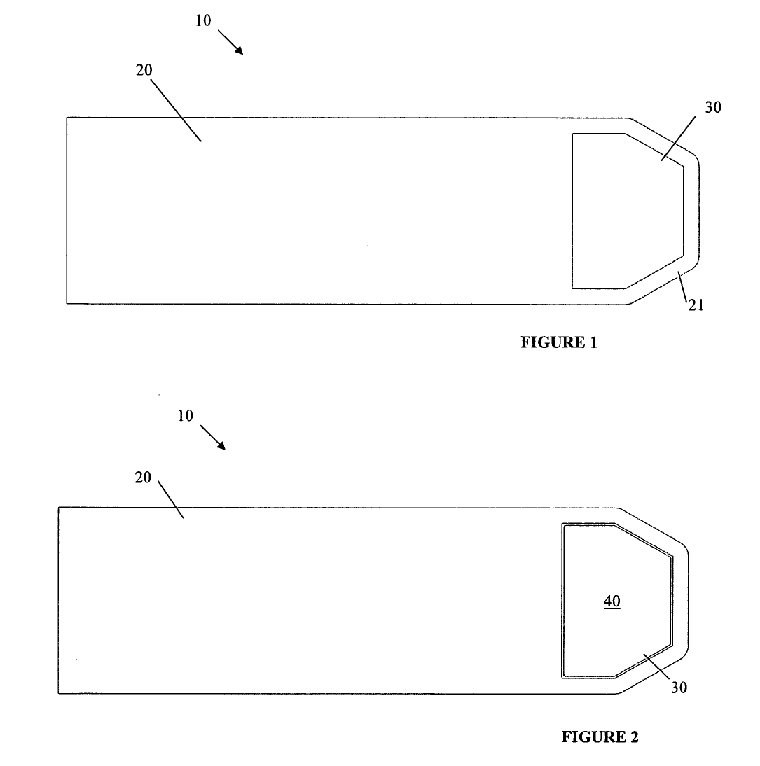

The present invention, as illustrated in FIG. 1 is an imaging table 10, preferably having a carbon fiber top 20. The table of the present invention allows proper positioning of a patient's head relative to the table top. While the table of the present invention is described herein as an imaging table, the table could be used for other processes which require proper positioning of a patient in a prone position, such as for surgery or therapy. As stated above, for imaging purposes, a carbon fiber top is used since it does not interfere with current imaging process. For other uses, the top 20 may be of materials based upon the needs and patient comfort. FIG. 1 illustrates a table having angled corners at the head end 21. Of course, tables may be of other shapes as well.

The table includes a large access hole 30 built into the head end. As illustrated in FIG. 2, various modular components or inserts 40 can be positioned within the access hole 30. Preferably, the modular components 40 are also manufactured out of carbon fiber to facilitate x-ray imaging. The modular components 40 provide a top surface that can be positioned at varying elevations relative to the plane of the table top. The varying elevations facilitate both correct positioning of the patient and breathing access.

In one embodiment of the invention, as illustrated in FIG. 2, the modular component 40 has a flat top surface which is coplanar with the table top when the modular component 40 is inserted in the access hole 30.

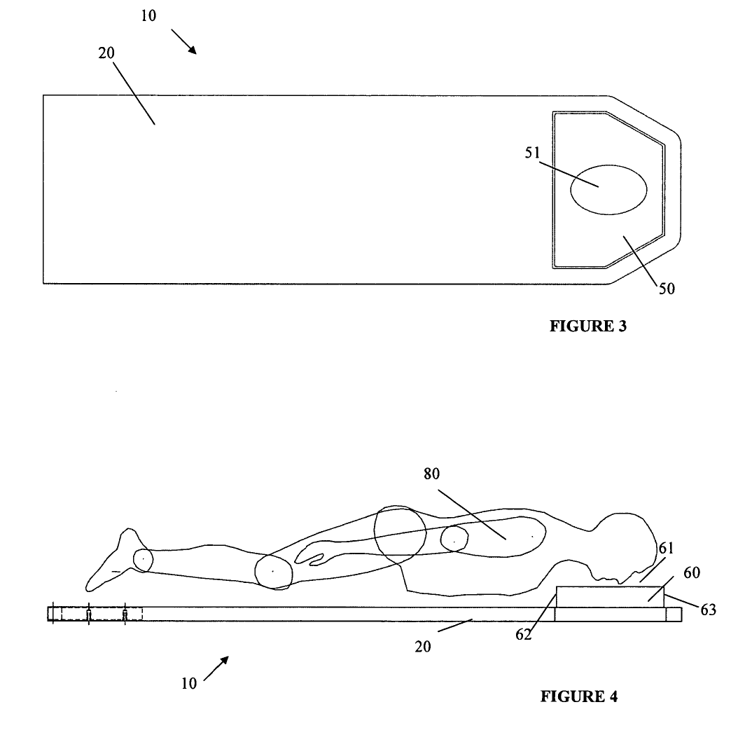

In another embodiment of the invention, as illustrated in FIG. 3, a modular component 50 includes an oval recess or hole 51 to accommodate the patient's face. The recess or hole 51 provides an opening so that the patient is able to more easily breath when in a face down position. Various modular components 50 may have different sized oval holes 51 to accommodate various sized patients.

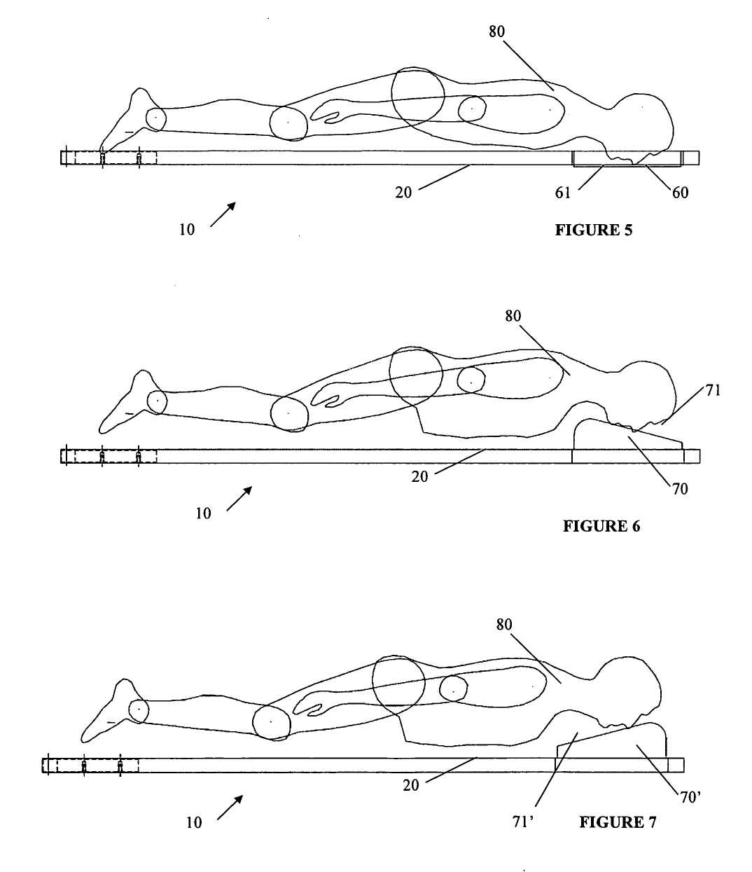

FIGS. 4 and 5 illustrate another embodiment of a modular component 60 which is reversible. The modular component 60 is in the form of a “tub” having a planar surface 61 and sides 62, 63. When placed in the access hole 30 of the table in a first position, illustrated in FIG. 4, the sides 62, 63 of the modular component 60 engage the sides of the access hole 30. The planar surface 61 extends above the plane of the table top 20. This allows the head of the patient 80 to be above the table top to properly position the back and neck. A flange (not shown) may be molded as part of the sides of the modular component 60 to facilitate the interaction with the sides of the access hole 30. As in the embodiment in FIG. 3, the planar surface 61 of the modular component 60 may also include an oval hole to accommodate the face of the patient to allow proper breathing.

When the modular component 60 is reversed, as illustrated in FIG. 5, it extends into the access hole 30. The planar surface 61 is now recessed within the access hole 30 so that the face of the patient 80 is positioned below the top of the table 20. This allows a different positioning of the back and neck of the patient for imaging purposes.

Other types and shapes of modular components can be used to properly position and align the head, neck, and spine of the patient for the desired imaging. As is known in the art, it may be desirable to have the patient's face angled. FIGS. 6 and 7 illustrate a modular component 70, 70′ which can be used to angle the patient's head and face. The modular component 70 is formed similar to the other modular components which extend above the top of the table. However, the upper surface 71 is angled so that the patient's face is similarly positioned. As is illustrated in FIG. 6, the upper surface 71 of the modular component may be higher towards the patient's neck and lower towards the end of the table. Alternatively, as illustrated in FIG. 7, the upper surface 71′ may be angled the opposite way so that it is higher towards the end of the table. If the access hole 30 and modular component 70 were symmetrical, a single modular component 70 could be used. The angle of the top could be changed by rotating the modular component 180 degrees. Of course, different modular components could have different heights.

The modular components 40, 50, 60, 70 have been illustrated with different configurations allowing positioning of the patient's head above, at and below the top of the table. The various heights above and below the top of the table depend upon the desired positioning of patients. Different modular components may be used with various heights or depths in order to accommodate variations in desired positions or in patient sizes.

Having disclosed at least one embodiment of the present invention, various adaptations, modifications, additions, and improvements will be readily apparent to those of ordinary skill in the art. Such adaptations, modifications, additions and improvements are considered part of the invention which is only limited by the several claims attached hereto.

1. A imaging table comprising:
a table top supported above the floor for supporting a patient to be imaged, the table top having an access hole positioned at a location corresponding to a location of the patient's head when supported on the table top; and
a modular component arranged to fit within the access hole, the modular component having an upper surface to support the patient's head when supported on the table top.

2. The imaging table of claim 1 wherein the modular component is arranged such that when positioned within the access hole, the upper surface of the modular component is substantially coplanar with an upper surface of the table top.

3. The imaging table of claim 1 wherein the modular component is arranged such that when positioned within the access hole, the upper surface of the modular component extends above an upper surface of the table top.

4. The imaging table of claim 3 wherein the modular component is arranged such that when positioned within the access hole, the upper surface is angled with respect to the upper surface of the table top.

5. The imaging table of claim 4 wherein the modular component is arranged such that when positioned within the access hole, the upper surface of the modular component is higher towards an end of the table top closest to the patient's head.

6. The imaging table of claim 4 wherein the modular component is arranged such that when positioned within the access hole, the upper surface of the modular component is lower towards an end of the table top closest to the patient's head.

7. The imaging table of claim 4 wherein the access hole has a symmetric shape and the modular component has a symmetric shape such that the modular component may be placed in the access hole in at least two different positions.

8. The imaging table of claim 1 wherein the modular component includes:
a bottom surface opposite the upper surface; and
a plurality of sides extending from the bottom surface; and
wherein the access hole and modular component are arranged such that when the modular component is placed within the access hole in a first position, the sides contact a portion of the access hole such that the upper surface is maintained above the upper surface of the table top.

9. The imaging table of claim 8, wherein the access hole and modular component are arranged such that then the modular component is placed within the access hole in a second position, the sides contact a portion of the access hole such that the patient's head is supported by the bottom surface positioned below the upper surface of the table top.

10. The imaging table of claim 1, wherein the modular component includes an indentation in a portion of the upper surface, the indentation being shaped to receive the patient's face.

11. The imaging table of claim 10, wherein the indentation is oval shaped.

12. The imaging table of claim 10 wherein the indentation extends through the modular component.

13. The imaging table of claim 1, wherein the table is formed of a radio translucent material.

14. The imaging table of claim 1, wherein the modular component is formed of a radio translucent material

15. An imaging table comprising:
a table top supported above the floor for supporting a patient to be imaged, the table top having an access hole positioned at a location corresponding to a location of the patient's head when supported on the table top; and
a plurality of modular components arranged to fit within the access hole, wherein the plurality of modular components include:
a first modular component having an upper surface to support the patient's head in a first position; and
a second modular component having an upper surface to support the patients head in a second position.

16. The imaging table of claim 15, wherein the first position is higher than the second position relative to the table top.

17. The imaging table of claim 15, wherein the first position is substantially coplanar with an upper surface of the table top.

18. The imaging table of claim 15, wherein the second position includes a different angle relative to the table top than the first position.
